(19) United States
(12) Patent Application Publication
(10) Pub. No.: US 2023/0260660 A1
(43) Pub. Date: Aug. 17, 2023

(54) VACCINE HESITANCY PREDICTION METHOD BASED ON EMERGENCY VACCINE

(71) Applicant: Zhengzhou University, Zhengzhou (CN)

(72) Inventors: Jian WU, Zhengzhou (CN); Quanman LI, Zhengzhou (CN)

(73) Assignee: Zhengzhou University, Zhengzhou (CN)

(21) Appl. No.: 18/140,648

(22) Filed: Apr. 28, 2023

(30) Foreign Application Priority Data
Sep. 22, 2022 (CN) 202211160437.2

(51) Int. Cl. G16H 50/30; G06N 20/00; G06N 7/01

(52) U.S. Cl. CPC G16H 50/30; G06N 20/00; G06N 7/01

(57) ABSTRACT

A vaccine hesitancy prediction method based on emergency vaccine is provided, including: collecting multi-dimensional variables of initial vaccine hesitancy influencing factors, screening the variables and constructing the model, and using the vaccine hesitancy prediction model to accurately identify high-risk groups of vaccine hesitancy. It can help to find the high-risk population of anti-vaccination, provide guidance for vaccination intervention, provide important technical support for the construction of “immune barrier”, and provide technical enlightenment for the prediction of vaccine hesitancy, which has certain economic and social benefits.

### CROSS REFERENCE TO THE RELATED APPLICATIONS

This application is based upon and claims priority to Chinese Patent Application No. 202211160437.2, filed on Sep. 22, 2022, the entire contents of which are incorporated herein by reference.

### TECHNICAL FIELD

The invention is related to the field of artificial intelligence technology, specifically a vaccine hesitancy prediction method based on emergency vaccine.

### BACKGROUND

Vaccine hesitancy has been defined as a lack of awareness of the safety and effectiveness of vaccines and diseases against which vaccine recipients may delay or refuse vaccination despite its availability, thus exposing themselves to preventable diseases. The World Health Organization (WHO) lists vaccine hesitancy as one of the top ten threats to global health; Vaccine hesitancy has become a major obstacle to the construction of “immune barrier”. Currently, there is still a lack of effective drugs and treatments for COVID-19. While public health interventions such as wearing masks, washing hands and maintaining safe social distancing play a very important role in controlling the epidemic, vaccination is the most cost-effective, safe and effective ways to prevent, control or even eliminate COVID-19. The COVID-19 vaccination programs in high-income countries provide scientific basis and practical experience for low—and middle-income countries, and also reflect on the emergence of vaccine hesitancy in the vaccination process in these countries. Therefore, it is the key to construct the emergency vaccine hesitancy prediction model to accurately identify high-risk groups of vaccine hesitancy, improve vaccination rate, and provide an effective path for the establishment of an “immune barrier” as soon as possible. However, few methods have been studied for predicting vaccine hesitancy of high-risk groups, so how to accurately predict vaccine hesitancy of high-risk groups? Providing important technical support for the construction of “immune barrier” has become a current research topic.

Therefore, the emergency vaccine hesitancy prediction method is urgently needed to address the aforementioned issues.

### SUMMARY

The invention aims to solve the defects existing in the prior art and propose a vaccine hesitancy prediction method based on the emergency vaccine.

In order to achieve the above purposes, the invention adopts the following technical solutions:

A novel vaccine hesitancy prediction method based on emergency vaccine consists of the following steps:

The influencing factors of vaccine hesitancy to be analyzed were collected. The variables of vaccine hesitancy influencing factors include gender, age, ethnicity, religious belief, marital status, education level, subjective social status, smoking status, drinking status, self-reported health status, chronic disease, prevention measures for COVID-19, SARS-CoV-2 skepticism, self-perceived risk of infection, self-perceived the possibility of cure, information sources of emergency vaccine, emergency vaccine skepticism, trust in doctors and vaccine developers, and convenience of COVID-19 vaccination.

The variables of vaccine hesitancy influencing factors to be analyzed were input into the pre-constructed vaccine hesitancy prediction model, so as to accurately identify the high-risk groups of vaccine hesitancy.

Further, the construction process of the vaccine hesitancy prediction model is as follows:

S1: Data collection and processing: the multi-dimensional initial variables of vaccine hesitancy influencing factors were collected and preprocessed. The multi-dimensions included basic demographic characteristics, health status, lifestyle, prevention measures for COVID-19, perception of COVID-19 epidemic, information sources of emergency vaccine, awareness of emergency vaccine and convenience of COVID-19 vaccination.

S2: Variables selection: univariate analysis was performed on the initial multi-dimensional influencing factors of vaccine hesitancy after pretreatment and then obtain the results, and potential covariates were screened based on them. Further, the covariates were analyzed by collinear analysis, and the covariates were listed as independent variables according to the results of collinear analysis.

S3: Model construction: The Logistic regression model was selected as the prediction model and constructed. At the same time, the independent variables were input as the training set to train the Logistic regression model, and the vaccine hesitancy prediction model was obtained.

S4: Model evaluation: the performance of the vaccine hesitancy prediction model was tested according to the degree of fit. If the vaccine hesitancy prediction model met the prespecified performance criteria, it would be output; Otherwise, we returned to step S3 for repeated training until the model met the prespecified performance criteria.

Further, the specific process of variables selection described in Step S2 is as follows:

S2.1: Obtain the multidimensional initial influencing factors of vaccine hesitancy, identify whether they belong to categorical variables, ordinal variables or continuous variables, assign corresponding values to various variables, and conduct univariate analysis to obtain the results of univariate analysis;

S2.2: According to the results of univariate analysis, the potential covariates in the multi-dimensional initial influencing factors of vaccine hesitancy with P value <0.05 were extracted;

S2.3: The least square method was used to regression the potential covariates to calculate the coefficient of determination R², and the variance inflation factor (VIF) was used to test whether there was collinearity among the potential covariates;

The specific formula for calculating the coefficient of determination R² is as follows:

$${r}^{2} = \frac{{Σ ( \hat{y} - \overline{y} )}^{2}}{{Σ ( y - \overline{y} )}^{2}} = 1 - \frac{{Σ ( y - \hat{y} )}^{2}}{{Σ ( y - \overline{y} )}^{2}}$$

$$\frac{{Σ ( \hat{y} - \overline{y} )}^{2}}{{Σ ( y - \overline{y} )}^{2}} :$$

the percentage of the variation in y value that has been explained by the regression relationship its total variation;

$$\frac{{Σ ( y - \hat{y} )}^{2}}{{Σ ( y - \overline{y} )}^{2}} :$$

the proportion of variation in y value that is not explained by the regression relationship in the total variation.

The formula for calculating the variance inflation factor VIF is as follows:

$${VIF}_{i} = \frac{1}{1 - {R}_{i}^{2}}$$

Where: Rᵢ is the multiple correlation coefficient of i^{th} variable xᵢ and all other variables xⱼ (i=1, 2 . . . k; i≠j).

Further, the characteristics of the Logistic regression model are as follows:

$$P ( y = 1 \vert x ) = f ( x ) = \frac{1}{1 + {e}^{- ( {w}_{0} + {w}_{1} {x}_{1} + {w}_{2} {x}_{2} + … + {w}_{n} {x}_{n} )}}$$

Where: w₀ is a constant; w₁ is the coefficient of x₁; w₂ is the coefficient of x₂; wₙ is the coefficient of xₙ.

Further, the degree of fit includes Fadden R², Cox&Snell R² and Nagelkerke R².

Compared with the prior art, the beneficial effects of the present invention are:

A vaccine hesitancy prediction method based on emergency vaccine proposed in this application is to obtain a vaccine hesitancy prediction model by collecting multi-dimensional variables of initial vaccine hesitancy influencing factors, screening the variables and building the model. The vaccine hesitancy prediction model of the present invention is used to accurately identify high-risk groups of vaccine hesitancy, which is helpful to assist in finding high-risk groups of vaccine hesitancy. It can provide guidance for vaccination intervention, important technical support for the construction of “immune barrier”, and technical enlightenment for the prediction of vaccine hesitancy, which has certain economic and social benefits.

### BRIEF DESCRIPTION OF THE DRAWINGS

The drawings are intended to provide further understanding of the invention and form part of the specification, are used in conjunction with embodiments of the invention to explain the invention, and do not constitute a limitation of the invention.

FIG. 1 shows the overall flow chart of a vaccine hesitancy prediction method based on emergency vaccine proposed by the invention.

FIG. 2 shows the flow chart of the construction of the emergency vaccine hesitancy prediction model based on vaccine hesitancy prediction method proposed by the invention.

### DETAILED DESCRIPTION OF THE EMBODIMENTS

Specific embodiments will be clearly and completely described in the following in conjunction with the drawings attached to the embodiments of the present invention, the technical solutions in the embodiments of the present invention, obviously, the described embodiments are only part of the embodiments of the present invention, not all embodiments.

In the description of the invention, it is to be understood that the bearings or positional relationships indicated by the terms “up”, “down”, “front”, “back”, “left”, “right”, “top”, “bottom”, “inside”, “outside”, etc., are based on the bearings or positional relationships shown in the accompanying drawings and are intended only to facilitate and simplify the description of the invention, It is not intended to indicate or imply that the device or element referred to must have a particular orientation, be constructed and operate in a particular orientation, and therefore cannot be construed as a limitation of the present invention.

According to the purpose of the invention, a diagnostic model was selected to estimate the risk of individual vaccine hesitancy, identify high-risk groups of vaccine hesitancy, and implement intervention measures as early as possible. Because the primary outcome was vaccine hesitancy, which was clearly a dichotomous outcome, a Logistic regression model was used to construct a prediction model.

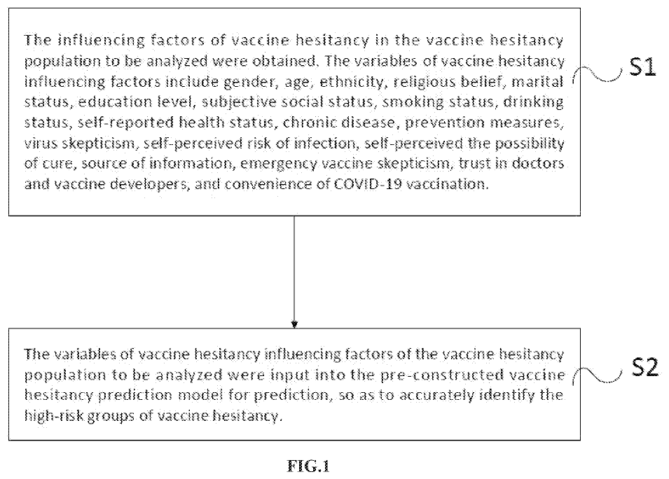

According to FIG. 1, this embodiment discloses the emergency vaccines based on vaccine hesitancy prediction method comprising the following steps:

The influencing factors of vaccine hesitancy in the vaccine hesitancy population to be analyzed were obtained. The variables of vaccine hesitancy influencing factors include gender, age, ethnicity, religious belief, marital status, education level, subjective social status, smoking status, drinking status, self-reported health status, chronic disease, prevention measures for COVID-19, SARS-CoV-2 skepticism, self-perceived risk of infection, self-perceived the possibility of cure, information sources of emergency vaccine, emergency vaccine skepticism, trust in doctors and vaccine developers, and convenience of COVID-19 vaccination.

The variables of vaccine hesitancy influencing factors of the vaccine hesitancy population to be analyzed were input into the pre-constructed vaccine hesitancy prediction model for prediction, so as to accurately identify the high-risk groups of vaccine hesitancy.

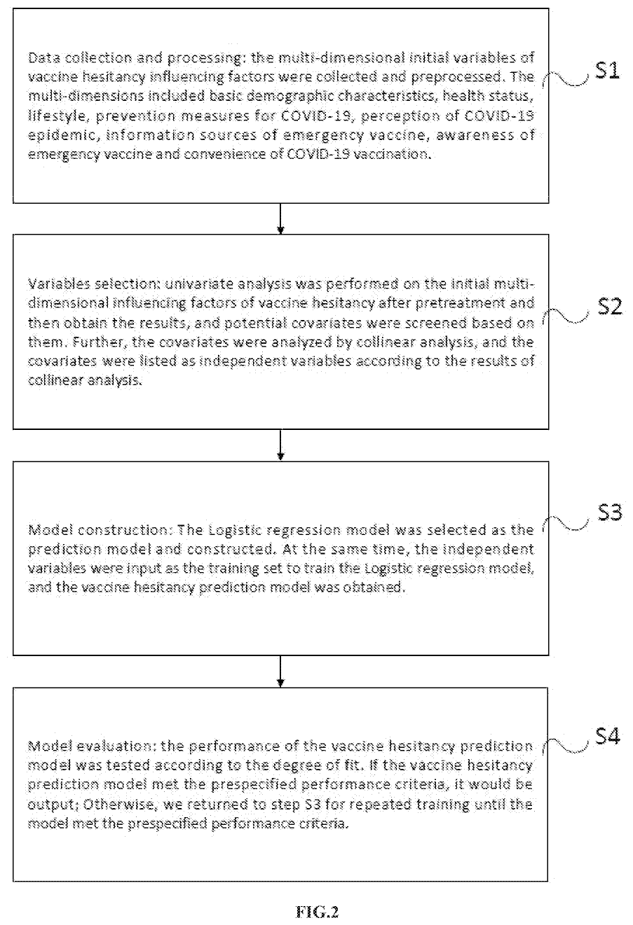

In one embodiment, referring to FIG. 2, the process of constructing the vaccine hesitancy prediction model is as follows:

S1: Data collection and data processing, multi-dimensional initial vaccine hesitancy influencing factors are collected and pre-processed; The multi-dimensions included basic demographic characteristics, health status, lifestyle, prevention measures for COVID-19, perception of COVID-19 epidemic, information sources of emergency vaccine, awareness of emergency vaccine and convenience of COVID-19 vaccination.

Specifically, the factors related to vaccine hesitancy were collected online, such as basic demographic characteristics, health status, lifestyle, prevention measures for COVID-19, perception of COVID-19 epidemic, information sources of emergency vaccine, emergency vaccine skepticism, trust in doctors and vaccine developers, awareness of emergency vaccine and convenience of COVID-19 vaccination. The samples that did not meet the target population were excluded. The basic demographic information included gender, age, ethnicity, religious belief, marital status, education level, subjective social status, occupation, medical insurance, economic income, etc. Health status included chronic disease, COVID-19 infection, and self-reported health status. Lifestyle included body weight, unhealthy diet, physical activity, sleep status, smoking, drinking, psychological stress, social support, physical examination. Prevention measures for COVID-19 included wearing masks, washing hands, maintaining safe social distancing. Perception of COVID-19 epidemic included self-perceived severity of the epidemic, self-perceived risk of infection, self-perceived possibility of cure. Awareness of emergency vaccine included the safety of emergency vaccine, the effectiveness of emergency vaccine, the validity period of emergency vaccine. trust in health system included trust in doctors and vaccine developers. Emergency vaccine hesitancy included vaccine hesitancy in the prime vaccination and vaccine hesitancy in the booster dose.

In particular, the data processing is used to check for missing values, outliers or outliers in each variable;” If the prevalence of chronic diseases has missing values, but the proportion of missing values is relatively low (missing variable <4%) without data imputation; There are abnormal values in economic income. In view of its sensitive information, this value is deleted.

S2: Variables selection: univariate analysis was performed on the initial multi-dimensional influencing factors of vaccine hesitancy after pretreatment and then obtain the results, and potential covariates were screened based on them. Further, the covariates were analyzed by collinear analysis, and the covariates were listed as independent variables according to the results of collinear analysis.

In one embodiment, the specific process of variable screening described in step S2 is as follows:

S2.1: obtain the multidimensional initial influencing factors, identify whether it is a categorical variable, a graded variable or a continuous variable, assign corresponding values to each type of variable, and conduct univariate analysis to obtain the results of univariate analysis.

Specifically, it also includes assigning a value to the categorical variable, the rank variable or the continuous variable, such as the gender variable belongs to the categorical variable; Educational level belongs to the grade variable; Age was a continuous variable. The categorical variables were assigned from 1 to 5, and dummy variables were set. The rank variables were assigned values from 1 to 5. The outcome variable (emergency vaccine hesitancy) was assigned a value of 0 (no hesitancy) and 1 (hesitancy). Finally, the distribution characteristics of each variable were evaluated and univariate analysis was performed, such as the χ2 test for categorical variables. The rank variables were analyzed by analysis of variance or rank sum test. Continuous variables were tested by t-test or Mann-Whitney U test.

S2.2: According to the results of univariate analysis, the potential covariates in the multi-dimensional initial influencing factors with P value <0.05 were extracted.

Specifically, the potential covariates of the Logistic regression model were screened according to the results of univariate analysis and previous studies. The covariates with P value <0.05 in the univariate analysis were entered into the initial multivariate model, and then the multivariate model was improved according to the backward method.

In one embodiment, according to the results of univariate analysis, it is shown that:

gender, age, ethnicity, religious belief, marital status, education level, subjective social status, smoking status, drinking status, self-reported health status, chronic disease, prevention measures for COVID-19, SARS-CoV-2 skepticism, self-perceived risk of infection, self-perceived the possibility of cure, information sources of emergency vaccine, emergency vaccine skepticism, trust in doctors and vaccine developers, and convenience of COVID-19 vaccination were included as independent variables in the initial multivariate model because the P value of univariate analysis was <0.05.

S2.3: The least square method was used to regression the potential covariates to calculate the coefficient of determination R², and the variance inflation factor (VIF) was used to test whether there was collinearity among the potential covariates;

The specific formula for calculating the coefficient of determination R² is as follows:

$${r}^{2} = \frac{{Σ ( \hat{y} - \overline{y} )}^{2}}{{Σ ( y - \overline{y} )}^{2}} = 1 - \frac{{Σ ( y - \hat{y} )}^{2}}{{Σ ( y - \overline{y} )}^{2}}$$

$$\frac{{Σ ( \hat{y} - \overline{y} )}^{2}}{{Σ ( y - \overline{y} )}^{2}} :$$

the percentage of the variation in y value that has been explained by the regression relationship in its total variation;

$$\frac{{Σ ( y - \hat{y} )}^{2}}{{Σ ( y - \overline{y} )}^{2}} :$$

the proportion of variation in y value that is not explained by the regression relationship in the total variation.

The formula for calculating the variance inflation factor VIF is as follows:

$${VIF}_{i} = \frac{1}{1 - {R}_{i}^{2}}$$

Where: Rᵢ is the multiple correlation coefficient of i^{th} variable xᵢ and all other variables xⱼ (i=1, 2 . . . k; i≠j).

In one embodiment, gender (VIF=1.20063), age (VIF=1.45801), ethnicity (VIF=1.06264), religious belief (VIF=1.16237), marital status (VIF=1.47302), education level (VIF=1.22772), subjective social status (VIF=2.83287; VIF=2.84501), smoking status (VIF=1.64292), drinking status (VIF=1.33679), self-reported health status (VIF=1.22787), chronic disease (VIF=1.23614), prevention measures for COVID-19 (masks (VIF=1.30773), hand washing (VIF=1.33214) and social distancing (VIF=1.23818), SARS-CoV-2 skepticism (VIF=1.90295), self-perceived risk of infection (VIF=1.22174), self-perceived possibility of cure (VIF=1.13797), and source of information (VIF=1.05) 211), emergency vaccine skepticism (VIF=2.07457), rust in doctors (VIF=2.43718), trust in vaccine developers (VIF=2.54409), and the convenience of COVID-19 vaccination (VIF=1.06694). All in the above were less than 4, indicating that there was no collinearity problem among the covariates.

S3: Model construction: Logistic regression model was selected as the prediction model and constructed. At the same time, the independent variables were input as the training set to train the Logistic regression model, and the vaccine hesitancy prediction model was obtained.

Specifically, the Logistic regression model is represented as follows:

$$P ( y = 1 \vert x ) = f ( x ) = \frac{1}{1 + {e}^{- ( {w}_{0} + {w}_{1} {x}_{1} + {w}_{2} {x}_{2} + … + {w}_{n} {x}_{n} )}}$$

Where: w₀ is a constant; w₁ is the coefficient of x₁; w₂ is the coefficient of x₂; wₙ is the coefficient of xₙ.

In one embodiment, using SAS 9.4, the above covariates were listed as independent variables, and the results of binary Logistic regression analysis with vaccine hesitancy or not as the outcome variable were as follows:

TABLE 1
Results of Logistic regression analysis.

| Variables | coefficient | standard error | Z value | P value |
|---|---|---|---|---|
| Gender | −0.01526 | 0.00311 | −4.92 | <.0001 |
| Age | −0.00822 | 0.00199 | −4.13 | <.0001 |
| Ethnicity | 0.03329 | 0.00712 | 4.67 | <.0001 |
| Religious belief | 0.03371 | 0.00432 | 7.80 | <.0001 |
| Marital status | 0.02110 | 0.00353 | 5.97 | <.0001 |
| Education level | −0.02664 | 0.00222 | −11.99 | <.0001 |
| Subjective social status | 0.00015419 | 0.00114 | 0.13 | 0.8927 |
| (China) | | | | |
| Subjective social status | 0.00414 | 0.00112 | 3.69 | 0.0002 |
| (community) | | | | |
| Smoking Status | −0.02053 | 0.00197 | −10.40 | <.0001 |
| Drinking status | 0.00070489 | 0.00173 | 0.41 | 0.6839 |
| Self-reported health | −0.00084832 | 0.00010863 | −7.81 | <.0001 |
| status | | | | |
| Chronic disease | 0.02787 | 0.00298 | 9.35 | <.0001 |
| Wearing mask | 0.14004 | 0.00586 | 23.91 | <.0001 |
| Washing hands | 0.05705 | 0.00405 | 14.08 | <.0001 |
| Maintaining safe social | 0.01018 | 0.00318 | 3.20 | 0.0014 |
| distancing | | | | |
| SARS-CoV-2 | 0.00013908 | 0.00022516 | 0.62 | 0.5368 |
| skepticism | | | | |
| Self-perceived risk of | −0.00674 | 0.00138 | −4.89 | <.0001 |
| infection | | | | |
| Self-perceived the | 0.01864 | 0.00133 | 13.99 | <.0001 |
| possibility of cure | | | | |
| Information sources of | 0.02984 | 0.00211 | 14.13 | <.0001 |
| emergency vaccine | | | | |
| Emergency vaccine | 0.01073 | 0.00184 | 5.84 | <.0001 |
| skepticism | | | | |
| Trust in doctors | 0.00009652 | 0.00196 | 0.05 | 0.9607 |
| Trust in developers | −0.02698 | 0.00282 | −9.58 | <.0001 |
| The convenience of | 0.06554 | 0.00490 | 13.38 | <.0001 |
| vaccination | | | | |
| Intercept | −0.21104 | 0.02244 | −9.41 | |

S4: Model evaluation: the performance of the vaccine hesitancy prediction model was tested according to the degree of fit. If the vaccine hesitancy prediction model met the prespecified performance criteria, it would be output; Otherwise, we returned to step S3 for repeated training until the model met the prespecified performance criteria.

Specifically, the degree of fit includes Fadden R², Cox&Snell R² and Nagelkerke R².

In one embodiment, the present invention makes use of the goodness-of-fit test for the performance of the prediction model, and the degree of fit of the Logistic regression model calculated from SAS: Fadden R²=0.24280, Cox&Snell R²=0.2048, Nagelkerke R²=0.2041.

The above is only a better specific implementation of the invention, but the scope of protection of the invention is not limited to this, any skilled personnel familiar with the field of technology in the scope of technology disclosed by the invention, according to the technical scheme of the invention and its inventive concept to be equivalent replacement or change, should be covered by the scope of protection of the invention.

What is claimed is:

1. A vaccine hesitancy prediction method based on an emergency vaccine comprising the following steps:
collecting vaccine hesitancy influencing factors and preparing to analyze, wherein variables of the vaccine hesitancy influencing factors comprises gender, age, ethnicity, religious belief, marital status, education level, subjective social status, smoking status, drinking status, self-reported health status, chronic disease, prevention measures for COVID-19, SARS-CoV-2 skepticism, self-perceived risk of infection, self-perceived the possibility of cure, information sources of emergency vaccine, emergency vaccine skepticism, trust in doctors and vaccine developers, and convenience of COVID-19 vaccination;
inputting the variables of the vaccine hesitancy influencing factors into a pre-constructed vaccine hesitancy prediction model to accurately identify high-risk groups of vaccine hesitancy.

2. The vaccine hesitancy prediction method based on the emergency vaccine described in claim 1, wherein a construction process of the pre-constructed vaccine hesitancy prediction model comprises the following steps:
S1: data collection and processing: collecting and preprocessing multi-dimensional initial variables of the vaccine hesitancy influencing factors, wherein the multi-dimensional initial variables comprises basic demographic characteristics, health status, lifestyle, prevention measures for COVID-19, perception of COVID-19 epidemic, information sources of the emergency vaccine, awareness of the emergency vaccine and convenience of COVID-19 vaccination;
S2: variables selection: performing a univariate analysis on the multi-dimensional initial variables of the vaccine hesitancy influencing factors after the preprocessing and then obtain results, and screening potential covariates, further, analyzing the potential covariates by collinear analysis, and listing the potential covariates as independent variables according to results of the collinear analysis;
S3: model construction: selecting and constructing a Logistic regression model as a prediction model, at the same time, inputting the independent variables as a training set to train the Logistic regression model, and then obtaining the pre-constructed vaccine hesitancy prediction model;
S4: model evaluation: testing a performance of the pre-constructed vaccine hesitancy prediction model according to a degree of fit, outputting the pre-constructed vaccine hesitancy prediction model if the pre-constructed vaccine hesitancy prediction model met a prespecified performance criteria, otherwise, returning to step S3 for repeating training until the pre-constructed vaccine hesitancy prediction model met the prespecified performance criteria.

3. The vaccine hesitancy prediction method described in claim 2, wherein in step S2, a specific process of the variables selection is as follows:
S2.1: obtaining the multi-dimensional initial variables of the vaccine hesitancy influencing factors, identifying a variable of the multi-dimensional initial variables of the vaccine hesitancy influencing factors, wherein the variable belongs to categorical variables, ordinal variables, or continuous variables, assigning corresponding values to the variable, and conducting the univariate analysis to obtain the results of the univariate analysis;
S2.2: according to the results of the univariate analysis, extracting the potential covariates in the multi-dimensional initial variables of the vaccine hesitancy influencing factors with P value <0.05;
S2.3: using a least square method to regression the potential covariates to calculate a coefficient of a determination R², and using a variance inflation factor (VIF) to test whether a collinearity relationship exists among the potential covariates;
a specific formula for calculating the coefficient of the determination R² is shown in formula (1):
$${r}^{2} = \frac{{Σ ( \hat{y} - \overline{y} )}^{2}}{{Σ ( y - \overline{y} )}^{2}} = 1 - \frac{{Σ ( y - \hat{y} )}^{2}}{{Σ ( y - \overline{y} )}^{2}} ; \qquad formula ( 1 )$$
$$\frac{{Σ ( \hat{y} - \overline{y} )}^{2}}{{Σ ( y - \overline{y} )}^{2}} :$$
a percentage of a variation in y value having been explained by a regression relationship in a total variation;
$$\frac{{Σ ( y - \hat{y} )}^{2}}{{Σ ( y - \overline{y} )}^{2}} :$$
a proportion of a variation in y value having not been explained by the regression relationship in the total variation;
a formula for calculating the variance inflation factor VIF is shown in formula (2):
$${VIF}_{i} = \frac{1}{1 - {R}_{i}^{2}} ; \qquad formula ( 2 )$$
wherein Rᵢ is a multiple correlation coefficient of i^{th} variable xᵢ and all other variables xⱼ (i=1, 2 . . . k; i≠j).

4. The vaccine hesitancy prediction method described in claim 2, wherein the Logistic regression model is shown in formula (3):
$$P ( y = 1 \vert x ) = f ( x ) = \frac{1}{1 + {e}^{- ( {w}_{0} + {w}_{1} {x}_{1} + {w}_{2} {x}_{2} + … + {w}_{n} {x}_{n} )}} ; \qquad formula ( 3 )$$
w₀ is a constant; w₁ is a coefficient of x₁; w₂ is a coefficient of x₂; wₙ is a coefficient of xₙ.

5. The vaccine hesitancy prediction method described in claim 2, wherein the degree of fit comprises Fadden R², Cox&Snell R², and Nagelkerke R².
